(19) United States
(12) Patent Application Publication
(10) Pub. No.: US 2025/0123169 A1
(43) Pub. Date: Apr. 17, 2025

(54) PRESSURE OUTPUT DEVICES FOR SENSING FLUID PRESSURE IN A FLUID PROCESSING SYSTEM

(71) Applicant: Fresenius Medical Care Holdings, Inc., Waltham, MA (US)

(72) Inventors: Peter M. Czechowicz, JR., Waltham, MA (US); James Brugger, Waltham, MA (US)

(21) Appl. No.: 18/380,298

(22) Filed: Oct. 16, 2023

(51) Int. Cl. G01L 19/00; A61B 5/00; A61M 1/16

(52) U.S. Cl. CPC G01L 19/0023; A61B 5/6866; A61M 1/1601

(57) ABSTRACT

A pressure output device (POD) assembly used for measuring fluid pressure in a fluid processing system is described. The fluid processing system may be or may include a dialysis machine. In one example, the POD assembly may include a connector configured to be coupled to a measurement port arranged on an outer surface of a fluid processing system, a POD receptacle configured to be installed on the outer surface over the connector, the POD receptacle may be configured to be coupled to the connector, and a POD may include an inlet port, an outlet port, and a sensor port, the POD may be configured to be removably installed within a POD seat formed in the POD receptacle, the sensor port may be arranged within a POD opening of the POD receptacle and coupled to the connector to fluidically couple the POD to the measurement port. Other embodiments are described.

### FIELD

The disclosure generally relates to devices and processes for facilitating pressure measurement within a fluid processing system, for example, a pressure output devices for an extracorporeal circuit of a dialysis system.

### BACKGROUND

Dialysis machines commonly monitor pressure in an extracorporeal blood circuit, for example, pressure from a blood chamber containing a blood-air interface. The blood chamber may be connected to a pressure port of the dialysis machine. A membrane or diaphragm may be positioned between the blood chamber and the pressure port. The membrane provides a sterile barrier to the blood circuit and prevents blood contamination of the machine, while allowing air pressure to pass through the membrane and act on the pressure transducer inside the machine. In some systems, the membranes may be configured as arterial and venous pressure monitor features or “pressure pods.” These pods may include a molded plastic feature integrated into the bloodline system to transmit pressure information from a blood side of the pod, across a thin diaphragm, to a pressure sensor or other pressure measurement feature of the machine.

Pressure monitoring components, such as membranes, pods, blood lines, and/or the like, require replacement, adjustment, and other maintenance. Conventional pressure monitoring components, particularly pressure pods, are difficult to remove, especially for patient, caregivers, and healthcare staff that are not specifically trained in machine maintenance techniques. For example, conventional pressure pods are typically connected to machine pressure ports via connection mechanisms that are challenging, if not impossible, for dialysis patients who have difficulty manipulating small, intricate connections (for instance, a luer lock mechanism).

It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In one example embodiment, a pressure output device (POD) assembly may include a connector configured to be coupled to a measurement port arranged on an outer surface of a fluid processing system; a POD receptacle configured to be installed on the outer surface over the connector, the POD receptacle configured to be coupled to the connector; and a POD that may include an inlet port, an outlet port, and a sensor port, the POD configured to be removably installed within a POD seat formed in the POD receptacle, wherein the sensor port is arranged within a POD opening of the POD receptacle and coupled to the connector to fluidically couple the POD to the measurement port.

In some examples of the POD assembly, the connector may include a covered-connection connector configured to be coupled to the measurement port; and a cover configured to be installed over the connection element.

In various examples of the POD assembly, the covered-connection connector may include at least one flange configured to engage at least one groove of the cover when the cover is installed over the covered-connection connector.

In some examples of the POD assembly, the connector may include a direct-connection connector having a POD end configured to engage the POD receptacle.

In various examples of the POD assembly, the direct-connection connector may include ridges configured to engage at least one engagement structure of the POD receptacle when the POD receptacle is installed over the direct-connection connector.

In some examples of the POD assembly, the connector may be configured to form a luer connection with the measurement port.

In various examples of the POD assembly, the POD receptacle may be configured to be coupled to the connector via a set screw fastener.

In some examples of the POD assembly, the POD may be configured to be installed within the POD receptacle by pushing the POD within the POD opening toward the fluid processing system until the POD is seated in the POD seat.

In various examples of the POD assembly, the POD may form an interference fit with the POD seat when installed in the POD receptacle.

In some examples of the POD assembly, the outlet port may be arranged in a top position, vertically above a bottom position of the inlet port.

In various examples of the POD assembly, fluid flow within a chamber may be in a rotary fluid flow rotating from the inlet port to the outlet port.

In some examples of the POD assembly, the fluid processing device may be a hemodialysis (HD) machine.

In various examples of the POD assembly, the measurement port may be a pressure port of a hemodialysis (HD) machine.

In some examples of the POD assembly, the POD may be fluidically coupled with a pressure sensor of a hemodialysis (HD) machine to provide pressure readings for arterial or venous blood flowing through an extracorporeal circuit.

In various examples of the POD assembly, the POD may include a flange configured to engage a membrane of the POD to form a dimple in the membrane to prevent vapor lock between the membrane and an inner surface of the POD.

In one example embodiment, a method of measuring pressure of blood flowing through an extracorporeal circuit of a dialysis machine may include installing a pressure output device (POD) that may include an inlet port, an outlet port, and a sensor port in a POD assembly, the POD assembly may include: a connector configured to be coupled to a pressure port arranged on an outer surface of the dialysis machine; and a POD receptacle configured to be installed on the outer surface over the connector, the POD receptacle configured to be coupled to the connector; wherein the POD is configured to be removably installed within a POD seat formed in the POD receptacle, wherein the sensor port is arranged within a POD opening of the POD receptacle and coupled to the connector to fluidically couple the POD to the pressure port; and measuring fluid pressure using a pressure sensor detecting movement of a membrane of the POD resulting from a flow of the blood through the POD.

In some examples of the method, the connector may include a covered-connection connector configured to be coupled to the measurement port; and a cover configured to be installed over the connection element.

In some examples of the method, the connector may include a direct-connection connector having a POD end configured to engage the POD receptacle.

In various examples of the method, the outlet port may be arranged in a top position, vertically above a bottom position of the inlet port.

In some examples of the method, fluid flow within a chamber may be in a rotary fluid flow rotating from the inlet port to the outlet port.

### BRIEF DESCRIPTION OF THE DRAWINGS

By way of example, specific embodiments will now be described, with reference to the accompanying drawings, in which:

FIG. 1 illustrates an exemplary extracorporeal circuit in accordance with the present disclosure;

FIGS. 2A and 2B illustrate an exemplary pressure output device (or pressure oscillating diaphragm) (POD) assembly in accordance with the present disclosure;

FIGS. 3A-3E illustrate an exemplary POD in accordance with the present disclosure;

FIGS. 4A-4D illustrate an exemplary covered-connection POD receptacle in accordance with the present disclosure;

FIGS. 5A-5D illustrate an exemplary direct-connection POD receptacle in accordance with the present disclosure;

FIGS. 6A-6D illustrate an exemplary covered-connection connector in accordance with the present disclosure;

FIGS. 7A-7C illustrate an exemplary covered-connection cover in accordance with the present disclosure;

FIGS. 8A-8C illustrate an exemplary direct-connection connector in accordance with the present disclosure;

FIGS. 9A-9D illustrate an exemplary covered-connection POD assembly in accordance with the present disclosure;

FIGS. 10A-10D illustrate an exemplary direct-connection POD assembly in accordance with the present disclosure;

FIGS. 11A-11C illustrate an exemplary POD assembly coupled to a dialysis machine in accordance with the present disclosure; and

FIGS. 12A-12C illustrate an exemplary flanged POD membrane in accordance with the present disclosure.

### DETAILED DESCRIPTION

The present embodiments will now be described more fully hereinafter with reference to the accompanying drawings, in which several exemplary embodiments are shown. The subject matter of the present disclosure, however, may be embodied in many different forms and should not be construed as limited to the embodiments set forth herein. Rather, these embodiments are provided so that this disclosure will be thorough and complete, and willfully convey the scope of the subject matter to those skilled in the art. In the drawings, like numbers refer to like elements throughout.

Various features of an improved fluid pressure monitoring system will now be described more fully hereinafter with reference to the accompanying drawings, in which one or more features of the fluid pressure monitoring system will be shown and described. It should be appreciated that the various features may be used independently of, or in combination, with each other. It will be appreciated that a dialysis-ultrasound system as disclosed herein may be embodied in many different forms and should not be construed as being limited to the examples set forth herein. Rather, these examples are provided so that this disclosure will convey certain features of the dialysis-ultrasound system to those skilled in the art.

The present disclosure describes a fluid pressure monitoring system for monitoring, measuring, or otherwise determining the pressure of fluid within a fluid processing system. In some embodiments, the fluid processing system may be a dialysis system, such as a hemodialysis (HD) system or a peritoneal dialysis (PD) system. In various embodiments, the fluid processing system may be or may include an extracorporeal circuit. The fluid pressure monitoring system may be configured to measure the pressure of blood within arterial and/or venous blood lines of an extracorporeal circuit.

Although a dialysis system, extracorporeal circuit, and/or blood pressure measurement is used in examples herein, these are for illustrative purposes only. Embodiments are not limited to use with dialysis systems, extracorporeal circuits, and/or blood pressure measurements as the pressure monitoring system and associated devices may be used to measure the pressure of various different types of fluids within numerous types of systems (including non-dialysis systems) that may operate with the components of the embodiments described in the present disclosure.

The pressure output device (or pressure oscillating diaphragm) (POD) assemblies of the present disclosure can be operatively coupled to the pressure ports of a dialysis machine, such as an HD machine, for use in measuring blood pressures during hemodialysis. The POD assemblies provide an airless system for transferring extracorporeal circuit pressures to pressure monitoring ports of the extracorporeal circuit, for example, and ultimately to pressure sensors or transducers within the dialysis machine. Each POD assembly includes a POD receptacle configured to be coupled to the pressure port of a dialysis machine. The POD receptacle is formed to receive a POD that includes an elastomeric diaphragm. Blood can flow through the POD, which provides positive or negative circuit pressure that displaces the diaphragm. The displacement of the diaphragm compresses or expands the volume of air between the diaphragm and the pressure transducer in the hemodialysis machine, with which the volume of air is in fluid communication. As the air volume changes, the resulting pressure will be detected by the pressure transducer. The POD assembly also protects the pressure transducer from blood contact, and provides a sterile barrier at the interface to the blood circuit.

Conventional pressure pod systems are difficult to install/uninstall or otherwise manipulate. For example, pressure pod systems are typically connected to dialysis machines, such as the pressure port of a dialysis machine, via small, intricate engagement mechanisms, such as fasteners, luer locks, and/or the like. The engagement mechanisms are challenging for technicians and, especially, dialysis patients who are prone to limited manipulation of small, intricate fasteners and other engagement elements.

Accordingly, POD assemblies according to various embodiments facilitate an easy push-pull for installing/uninstalling (or load-unload) of PODs, while providing a stable, strong connection to the dialysis machine. In addition, POD assemblies are configured to ensure that the pod can only be installed in a single (correct) orientation. In some examples, for ease of loading and unloading, the sensing port in POD top or cap of the POD assembly has been positioned (e.g., rotated 180 degrees compared with previous configurations) to be closer to the inlet/outlet ports on the pod bottom or base. This allows the use of the tubing coming out of the ports in the POD base to be used to unload the POD from the POD assembly. For example, the sensor port being in this position decreases the stress on the port during loading and unloading of the POD. Furthermore, POD receptacles are configured to ensure that any technician, patient, and/or the like can easily make the machine modification to allow this receptacle to be added to an existing machines without requiring modification to the machine and/or sending the machine to a manufacturer and/or the like for rework.

POD assemblies according to exemplary embodiments may be used with various types of dialysis machines. A non-limiting example of a dialysis machine is the 2008T BlueStar® or 5008 dialysis machine provided by Fresenius Medical Care North America of Waltham, Massachusetts, United States of America. POD assemblies according to exemplary embodiments may be used with other dialysis machines. In general, a POD assembly may be used with any machine that includes a port or other engagement interface capable of being coupled to a POD receptacle.

In some embodiments, a POD receptacle may be mounted to the dialysis machine by a dialysis technician, patient, and/or the like. In some embodiments, the POD receptacle may be coupled to an engagement element of the dialysis machine. A non-limiting example of an engagement element may be or may include a luer interface (for instance, a twist fit or twist-and-lock luer interface). Although a luer interface is used in some examples, embodiments are not so limited, as the POD receptacle (and associated connection elements) may engage with various other types of engagement elements, such as threaded elements, pressure-fit elements, interference-fit elements, and/or the like. In some embodiments, engagement element may provide a hermetic seal between a port of the machine and the POD.

In one example, a seal with female luer interface is attached to an existing male luer of the pressure monitor port on the face of the machine. An inner adapter is then slid over the female luer and secured via a set screw that engages the metal outer portion of the male luer on the machine. The female luer may include wings, flanges, and/or other projections that are received in respective slots in the inner adapter such that the luer connection is secured from inadvertent disconnection. The inner adapter may include an opening to expose the end of the seal for connection to the pressure transmission port of the POD. A receptacle or outer adapter, with recesses corresponding to the geometry of the pressure pods is then slid over the inner adapter and secured via a set screw that engages the outer surface of the inner adapter. In some embodiments, the outer surface of the inner adapter may be cylindrical, such that the orientation of the wings of the female luer do not have an effect on the orientation of the receptacle. The receptacles may have a geometry that requires the PODS to be inserted in the correct orientation, with the outlet ports above the inlet ports. In this manner, the POD assembly interface does not entail any irreversible changes, such as drilling holes, to the dialysis machine and allows for easy removal for replacement of the seal attached to the pressure monitor port.

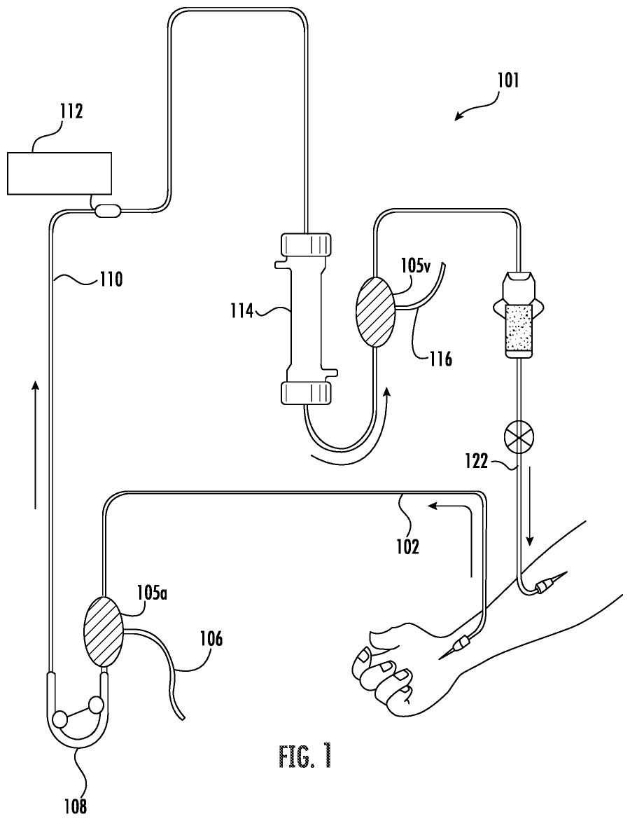

FIG. 1 illustrates an exemplary extracorporeal circuit in accordance with the present disclosure. More specifically, FIG. 1 is a schematic view of an extracorporeal blood circuit 101 for administration of hemodialysis. From a patient, a first arterial tubing 102 carries blood to a POD assembly 105 (for example, an arterial POD assembly 105a. A pressure tubing 106, connected to the sensor port of arterial POD assembly 105a, directs a pressure output from POD assembly 105a to an arterial pressure port (not shown) of the hemodialysis machine. Blood flows through the POD assembly 105a to a blood pump 108, for example, a peristaltic blood pump. From blood pump 108, blood is moved through a tubing 110 to a dialyzer 114. Along tubing 110 an additional pump 112 (such as a syringe pump) may be provided in fluid communication with tubing 110. Pump 112 may be a heparin pump and may be configured to inject heparin into blood circuit 101. For the sake of simplification, dialysate lines and a dialysate circuit are not shown connected to dialyzer 114.

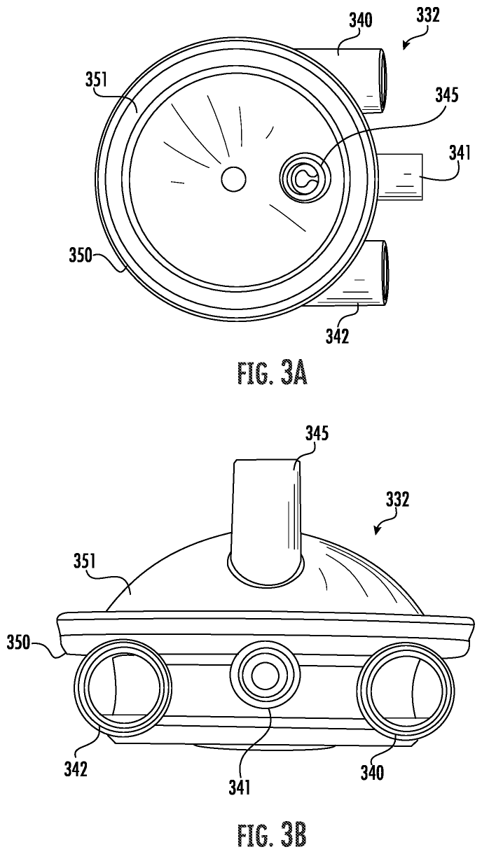

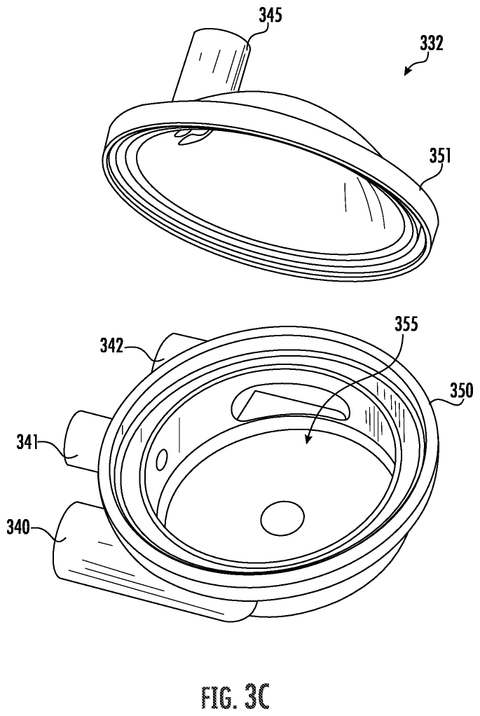

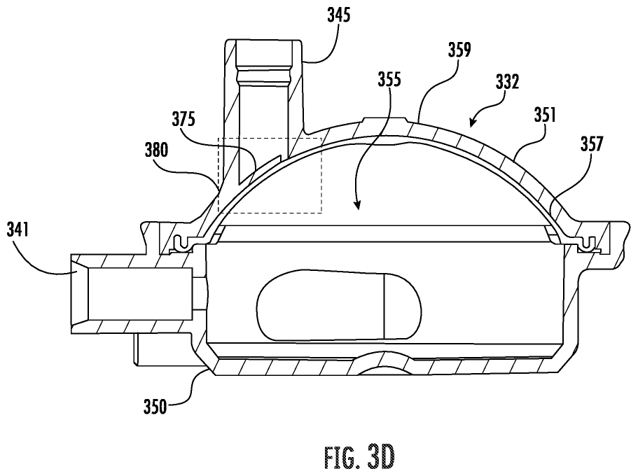

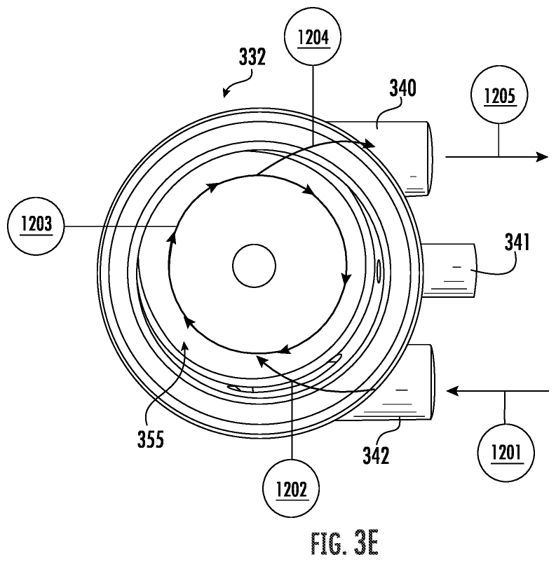

Blood exiting dialyzer 114 travels through another segment of tubing to a venous POD assembly 105v. A pressure tubing 116 in fluid communication with the sensor port of POD assembly 105v carries a pressure output to a venous pressure port (not shown) of the hemodialysis machine. Although FIG. 3 shows exemplary positions for arterial POD assembly 105a and venous POD assembly 105v, it should be understood that the POD assemblies can be arranged at different locations along blood circuit 101.

Blood flowing through the venous POD assembly 105v exits POD assembly 105v and is carried along another segment of tubing 322 that may include additional devices and elements, such as an air trap, an air detector 318, and/or the like.

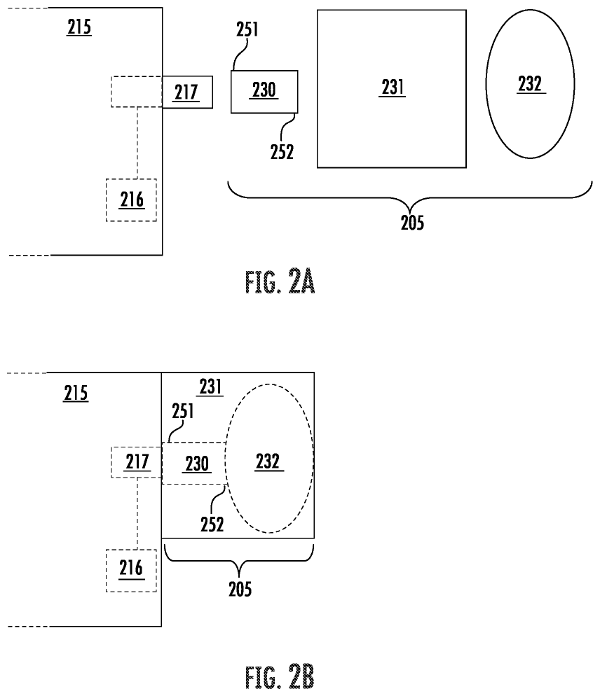

FIGS. 2A and 2B illustrate an exemplary POD assembly in accordance with the present disclosure. More specifically, FIG. 2A depicts a POD assembly 205 disconnected from a fluid processing device 215 and FIG. 2B depicts POD assembly 205 coupled to fluid processing device 215. Fluid processing device 215 may include a pressure sensor 216 coupled to a pressure port 217. POD assembly 205 may include a POD receptacle 231 configured to receive a POD 232.

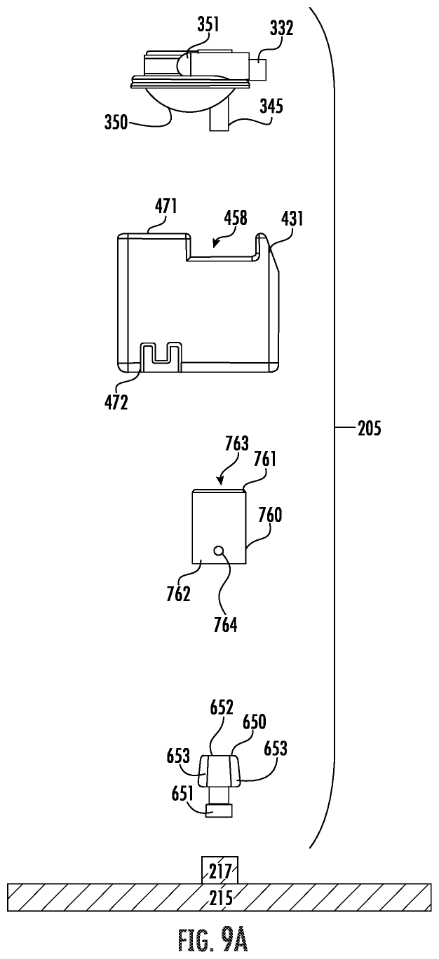

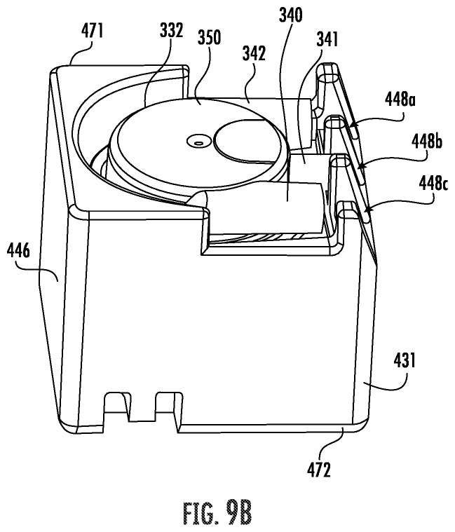

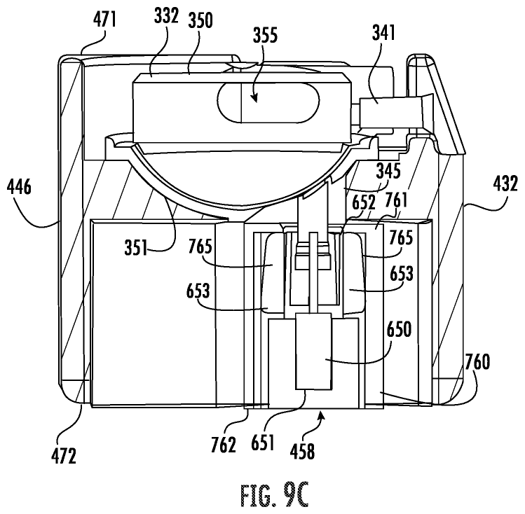

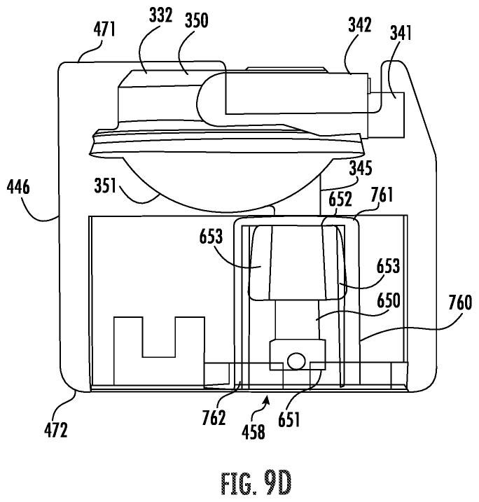

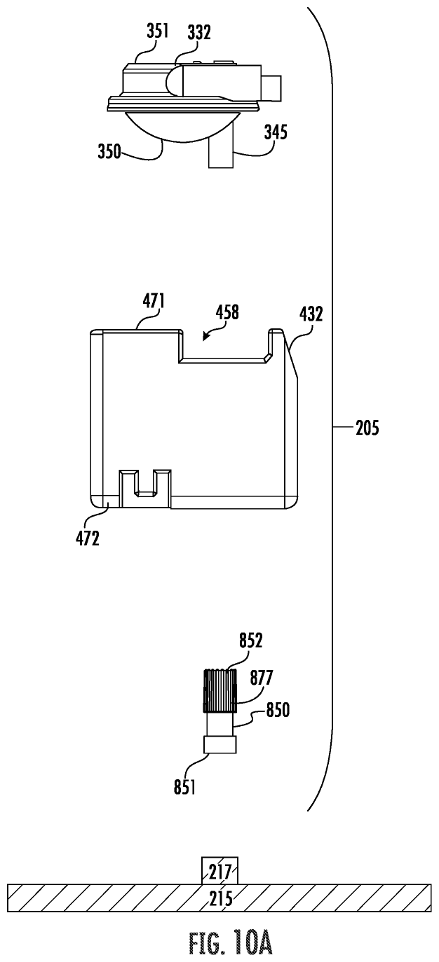

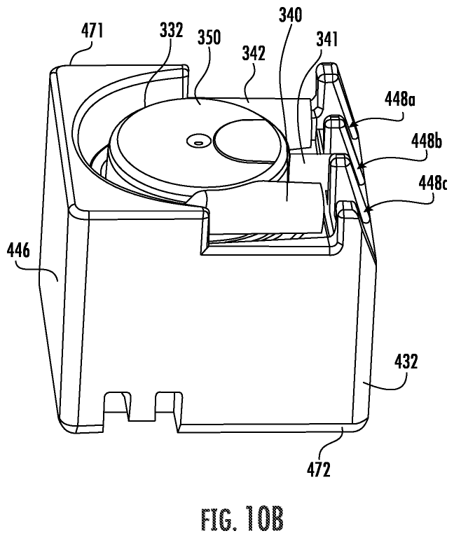

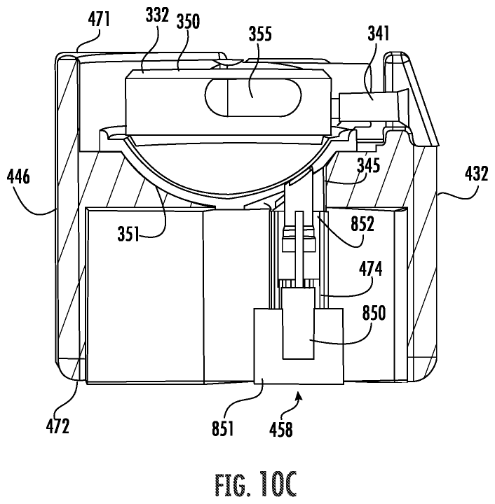

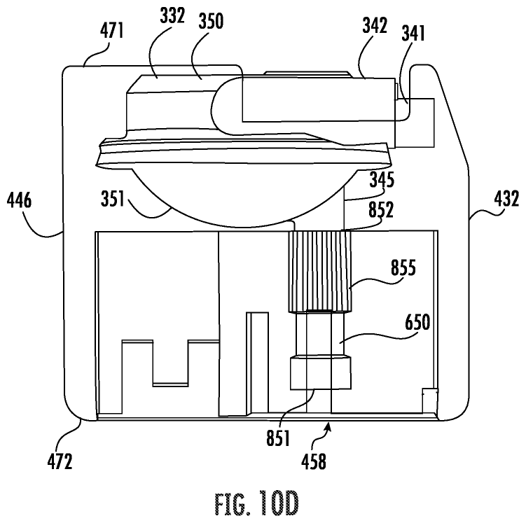

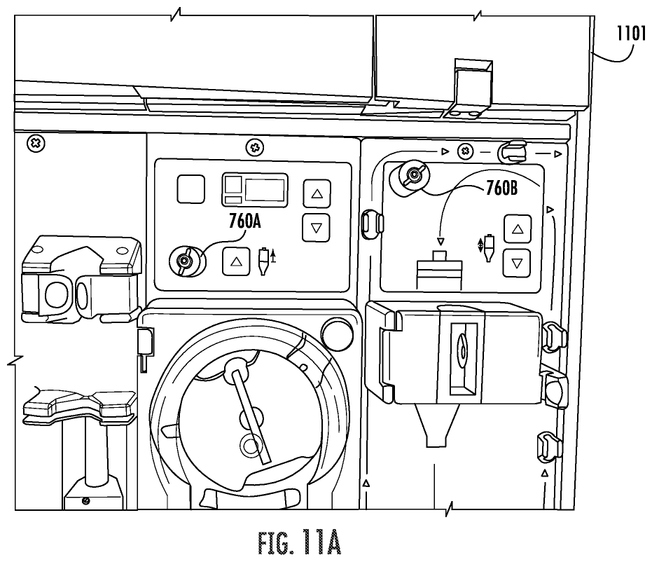

In some embodiments, a connection element 230 may have a first or machine end 251 configured to engage with pressure port 217 and a second or POD end 252 configured to engage with a POD 232. In various embodiments, connection element 230 may be configured to provide an attachment mechanism for POD receptacle 230 to attach to pressure port 217. In some embodiments, connection element 230 may include a plurality of elements (see, for instance, FIGS. 9A-9D). For example, connection element 230 may include a fixation element configured to connect with a corresponding component of pressure port, such as a luer fitting, and a cover element configured to be arranged over the fixation element. The cover element may be configured to engage with a corresponding structure of POD receptacle 230. In various embodiments, connection element 230 may be formed of a single element (see, for instance, FIGS. 10A-10D). For example, connection element 230 may be a single element with the machine end 251 configured as a luer locking element and the POD end 252 configured to engage a portion of POD receptacle 231 and/or POD 232. Connection element 230 may form a sealable connection with POD 232 so that POD 232 and port 217 are fluidically coupled.

In some embodiments, POD receptacle 231 may be coupled to connection element 230 via one or more fasteners. For example, POD receptacle 231 may include a set screw interface for installing a set screw that couples POD receptacle 231 to connection element 230.

FIGS. 3A-3E illustrate an exemplary POD in accordance with the present disclosure. More specifically, FIGS. 3A-3D depict various views of a POD, including a top-down view (FIG. 3A), a front view (FIG. 3B), a perspective, exploded view (FIG. 3C), and a cross-sectional view (FIG. 3D). FIG. 3E depicts fluid flow through the POD depicted in FIGS. 3A-3D.

In general, unless otherwise noted, for components of a POD assembly, “top” or a top orientation refers to an end or side that faces away (or is distal to) a machine where the POD assembly is installed; “bottom” or a bottom orientation refers to an end or side that faces toward (or is proximal to) a machine where the POD assembly is installed. For the inlet/outlet ports (340 and 342), the “top” refers to being higher or above, from a vertical perspective, a “bottom” component (e.g., inlet port 340) is arranged on the bottom (closer to the ground or floor) and outlet port 342 is arranged on top (above inlet port 340).

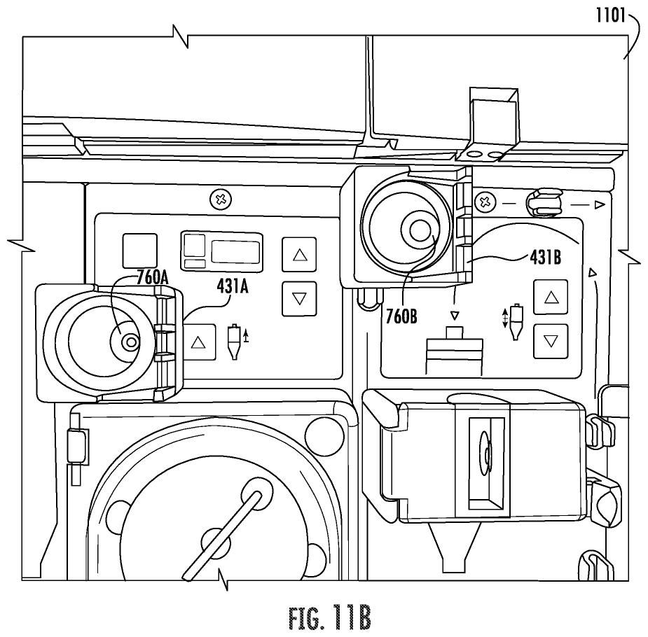

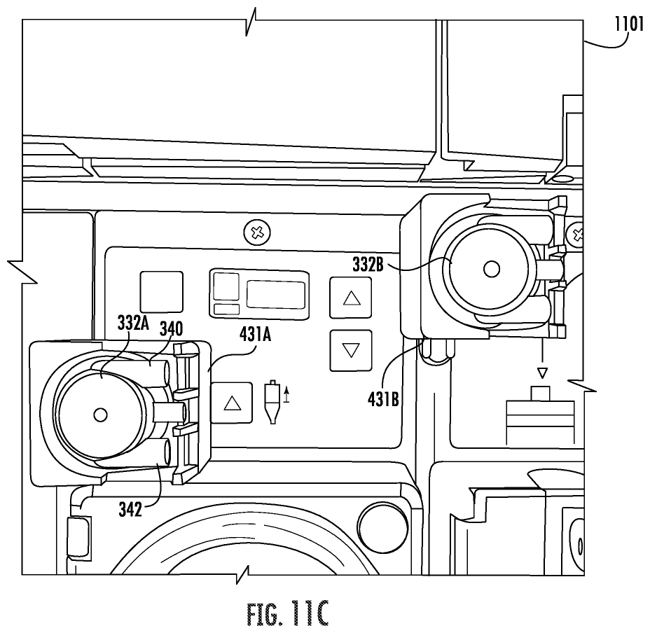

As shown in FIGS. 3A-3D, a POD 332 may include a cap (or top) 351, a membrane or diaphragm 357, and a base (or bottom) 350, assembled together. A fluid inlet port 340 and a fluid outlet port 342 are formed on the base 350. When installed, outlet port 340 is arranged to be on top (e.g., higher vertically) and inlet port 342 is arranged to be on the bottom (see, for example, FIG. 11A), for example, to help clear air out of POD 332 with inlet port 342 on the bottom and outlet port 340 on the top. Outlet port 340 and inlet port 342 may be configured to interface with a fluid line, such as flexible tubing (e.g., 4.55 mm×6.71 mm tubing).

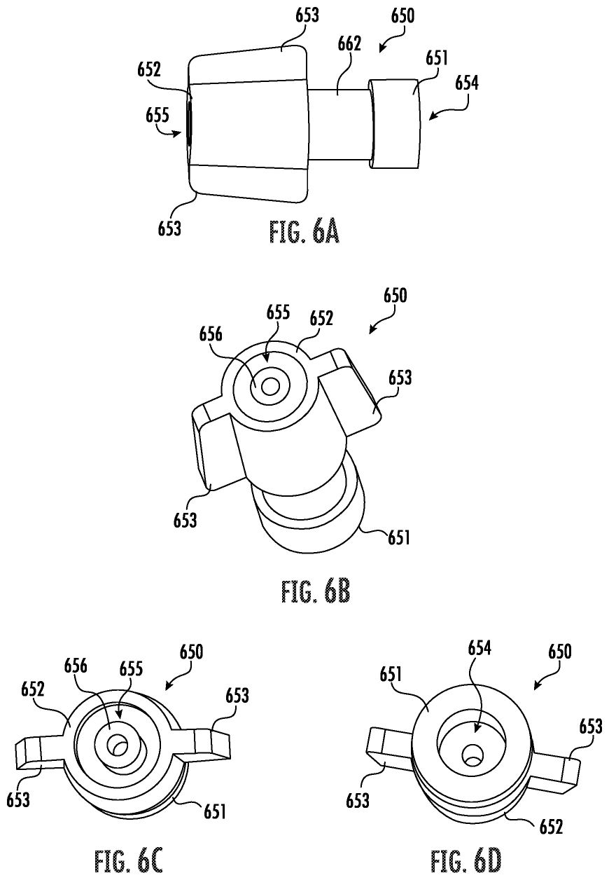

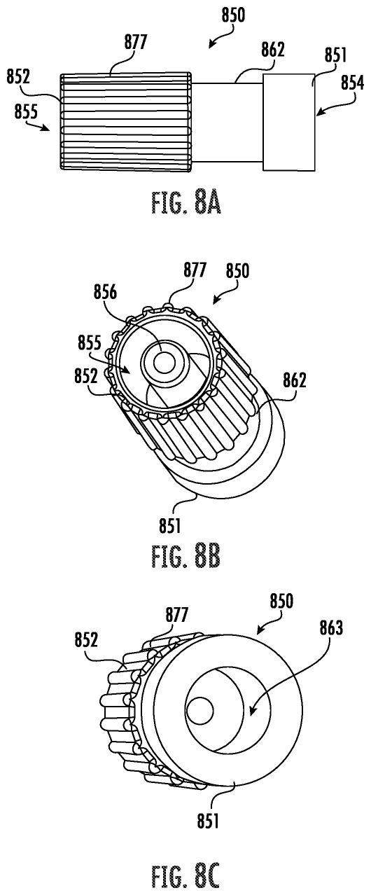

A sensor port 345 may be arranged in cap 351 for coupling POD 332 to a pressure port of a dialysis machine. Sensor port 345 may be configured to interface with one or more connection elements (see, for example, FIGS. 9A-9D and/or 10A-10D) to form a fluid connection between POD 332 and the pressure port of a dialysis machine. Sensor port 345 may be fluidically coupled to the pressure port (for example, via a connector, such as connector 650 of FIGS. 6A-6D and/or connector 850 of FIGS. 8A-8C).

In some embodiments, membrane 357 is formed against cap 351. For example, cap 351 and diaphragm may be part of the same two-shot mold so that membrane 357 is formed against cap 351. In various embodiments, cap 351 and membrane 357 may be permanently or semi-permanently coupled to base 350. For example, cap 351 and membrane 357 may be ultrasonically welded to base 350. A fluid chamber 355 may be formed between base 350 and membrane for the flow of fluid within POD 332.

POD 332 and components thereof, such as base 350 and cap 351, may be formed of various materials. In some embodiments, all or a portion of POD 332 may be formed of materials to form POD 332 as semi-rigid, translucent, gamma stable, solvent bondable, and/or the like. In some embodiments, POD 332 and/or portions thereof may be formed of one or more polymers, MABS transparent polymers, Terlux®, injection mold grade polymers, combinations thereof, and/or the like. In some embodiments, membrane 357 may be formed of various materials to provide a flexible and/or translucent component. In some embodiments, membrane 357 and/or portions thereof may be formed of one or more polymers, elastomers, thermoplastic elastomers, Medalist®, combinations thereof, and/or the like.

In some embodiments, an auxiliary port 341 may be formed in base 351. In various embodiments, an auxiliary port 341 may not be formed in base 351. Auxiliary port 341 may be configured to receive certain fluids, such as saline, heparin, and/or the like for inflow into POD 332, into an extracorporeal circuit, and into a patient. In some embodiments, a POD 332 may be designated as an arterial POD, for the arterial circuit, or a venous POD, for the venous circuit. In some embodiments, an auxiliary port 341 may be included in an arterial POD and excluded from a venous POD.

As shown in FIG. 3E, during operation, fluid inflow 1201 of a fluid (for instance, blood of a dialysis patient) may flow into fluid chamber 355 of POD 332 via inlet port 342. Input fluid flow 1202 may enter a circular, angular, swirling, or rotary fluid flow 1203 within chamber 355. Output fluid flow 1204 may exit chamber 355 via outlet port 340 and fluid outflow 1205 may proceed through the extracorporeal circuit. The positioning and orientation of outlet port 340 (being on the top) and inlet port 342 (being on the bottom) combined with the shape or form of chamber 355 creates rotary fluid flow 1203. Rotary fluid flow 1203 may facilitate the rapid movement of all of the fluid within chamber 355, for instance, reducing or even completely eliminating fluid flow “dead zones.” Conventional pressure pods or similar devices do not facilitate rotary fluid flow. Accordingly, fluid flows slowly or only in certain areas, causing inadequate fluid flow through the extracorporeal circuit (for instance, blood may become trapped within flow dead zones) and pressure reading inaccuracies. The configuration of PODs 332 and components thereof (e.g., input/output ports) ensure proper filling, air clearance, and the desired swirling of fluid.

As the fluid flows through chamber 355, the fluid exerts a pressure on membrane 357. The pressure may be positive or negative. The pressure causes membrane 357 to move or remain toward (positive pressure) or away (negative pressure) the pressure port of the dialysis machine where POD 332 is installed. Because POD 332, particularly sensor port 345, is fluidically coupled to the pressure port, the movement of membrane 357 is detected by the pressure sensor of the machine and is translated to a pressure reading. For example, the movement of membrane 357 causes a corresponding change in a pressure of a pressure fluid (for instance, air) within or in fluid communication with the pressure port. The change in the pressure fluid is detected by the machine pressure transducer.

In various embodiments, POD 332 may be coupled to a machine by being installed within a POD receptacle affixed to the machine. POD receptacles may be affixed to a machine using various connection elements and techniques.

In one example, a cover-connection may be configured in which a POD connector is coupled to a corresponding machine connection element and a cover element is positioned over the POD connector. In general, a POD connector is configured to provide a fluid connection between the POD and the pressure port of a machine. In one embodiment, the POD connector and the machine connection element may be corresponding luer fittings. For example, POD connector may be a female luer fitting and the machine connection element may be a male luer fitting. The cover element may be coupled to the POD connector, for instance, via a friction fit, locking fit, and/or a set screw or other fastener. The POD receptacle may be positioned over the cover element and secured to the cover element via various coupling techniques, such as a friction fit, locking fit, and/or a set screw or other fastener. In another example, a direct-connection may be configured in which the POD receptacle engages directly with the POD connector (i.e., no cover element is used).

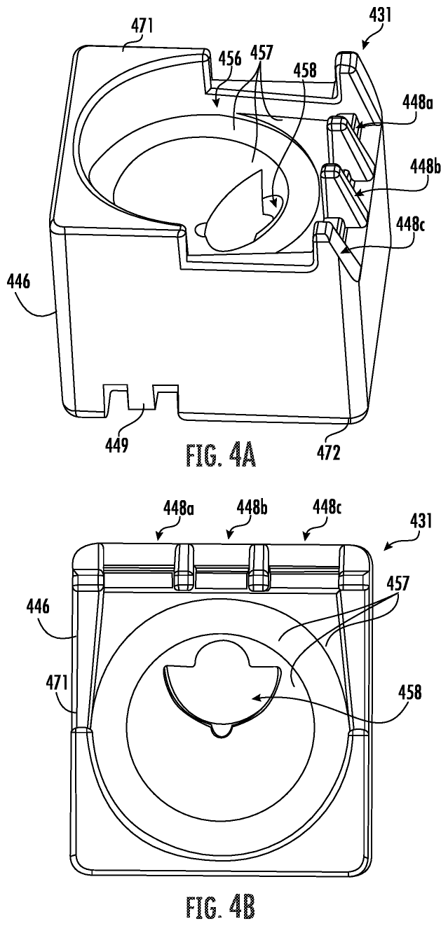

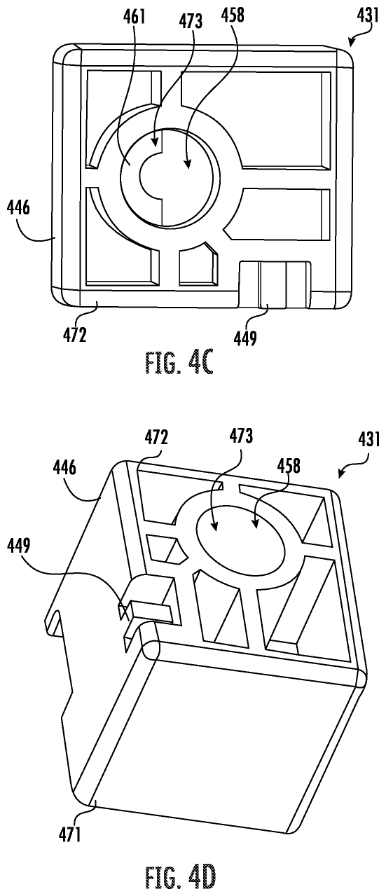

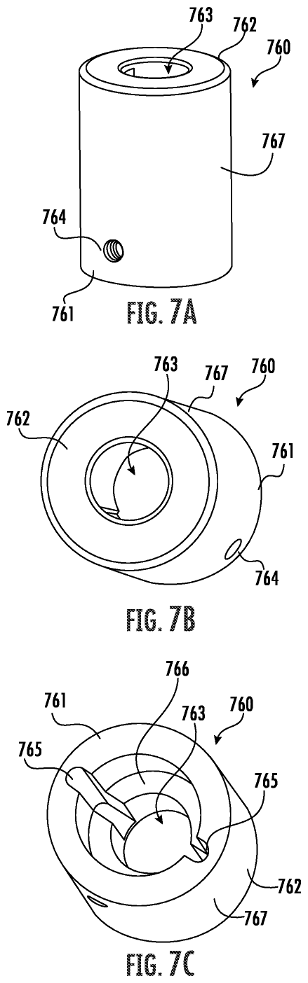

FIGS. 4A-4D illustrate an exemplary covered-connection POD receptacle in accordance with the present disclosure. More specifically, FIGS. 4A-4D depict various views of a covered-connection POD receptacle, including a side perspective view (FIG. 4A), a top view (FIG. 4B), a bottom view (FIG. 4C), and a bottom perspective view (FIG. 4D). In some embodiments, the covered-connection POD receptacle of FIGS. 4A-4D may be configured to operate with the POD connector of FIGS. 6A-6D and/or the cover element of FIGS. 7A-7C.

As shown in FIGS. 4A-4D, covered-connection POD receptacle 431 may include a housing 446 having a bottom or machine side 472 and a top or POD side 471. In some embodiments, housing 446 may be cubed or generally cubed shaped. When installed on a machine, machine side 472 may engage the machine and POD side 471 may face outward (i.e., away from the machine) and may be configured to receive POD 332. POD side 471 may include a POD opening 456 and a POD seat 457 configured to receive POD 332, particularly base 350 of POD. In some embodiments, POD opening 456 and POD seat 457 may be shaped, sized, or otherwise configured to correspond to or mate with POD 332. In some embodiments, POD opening 456 and/or POD seat 457 may be configured to facilitate a press, friction, interference, and/or the like fit with POD 332. For example, POD 332 may be held within POD receptacle 431 after being arranged within POD opening 456 and seated against POD seat 457. In various embodiments, POD seat 457 may be or may include portions that are rounded to allow for POD 332 to be unloaded by pulling on tubing coupled to POD 332 (e.g., tubing connected to one or more of ports 340-341).

POD receptacle 431 may include a sensor port opening 458 for sensor port 345 of POD 332 to extend through when installed within POD opening 456. One or more channels 448a-c may be formed in POD side 471 that are configured to allow ports and/or tubing to extend through a top portion of housing 446.

In some embodiments, POD receptacle 431 may include one or more engagement structures, features, or cutouts 449 configured to engage one or more elements arranged on an external surface of machine. Engagement structures 449 may be configured to provide clearance for existing components (e.g., buttons, lights, and/or the like) on the external surface of a machine where POD receptacle 431 is being installed. In various embodiments, engagement structures 449 may be configured to engage a machine component to prevent motion of POD receptacle 431, such as rotation. For example, engagement structure 449 may be configured to correspond to the shape of a component on the external surface of a machine so that, when POD receptacle 431 is installed on the external surface of the machine, engagement structure is arranged over the component, which thereby prevents rotation of POD receptacle.

The alignment of POD receptacle 431 may be constrained by features on a particular machine, for example, that engage with engagement structure 449. For an arterial POD 332, the bottom of POD receptacle 431 may align with the pump housing and prevent rotation (for instance, for a Fresenius 5008 dialysis machine). For a venous POD 332, the bottom of POD receptacle 431 may have a rectangular engagement structure or cutout 449 that aligns over a tube snap on the front face (for instance, for a Fresenius 5008 dialysis machine) which prevents rotation.

Referring to FIGS. 4C and 4D, a cover element opening 473 may be arranged in machine side 472. Cover element opening 473 may be configured to receive a cover element (see, for example, FIGS. 7A-7C or 9A-9D). In some embodiments, a seat 461 may be arranged within cover element opening 473 for engaging a top portion of a cover element when installed over the cover element.

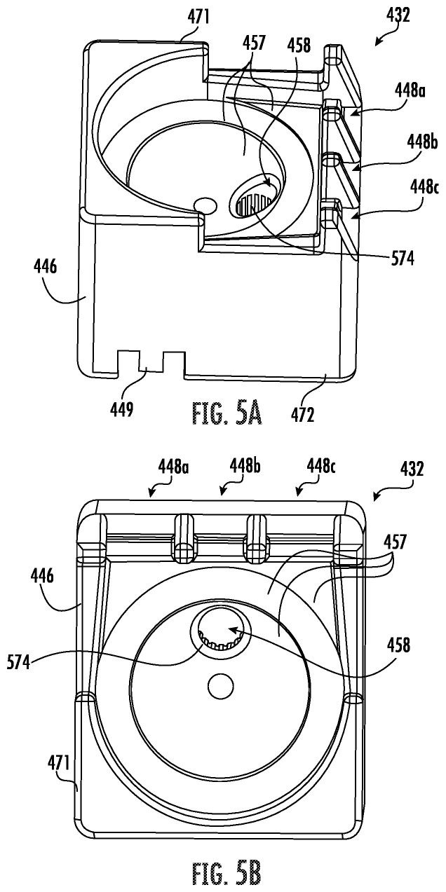

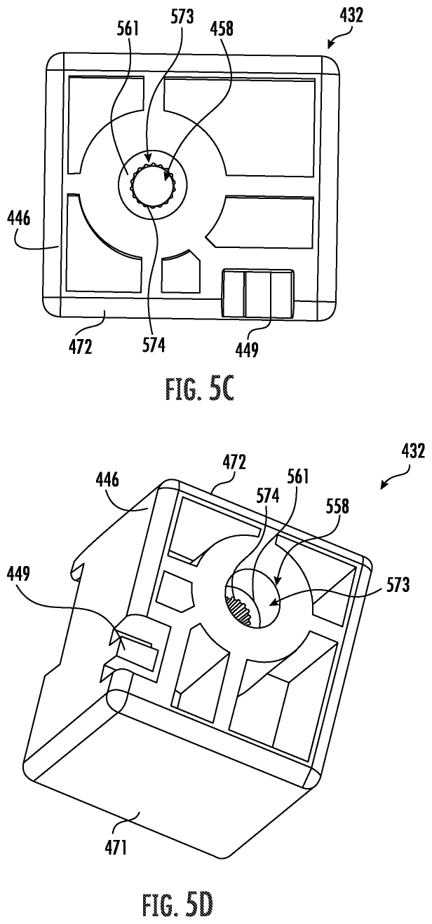

FIGS. 5A-5D illustrate an exemplary direct-connection POD receptacle in accordance with the present disclosure. More specifically, FIGS. 5A-5D depict various views of a direct-connection POD receptacle, including a side perspective view (FIG. 5A), a top view (FIG. 5B), a bottom view (FIG. 5C), and a bottom perspective view (FIG. 5D). In some embodiments, the covered-connection POD receptacle of FIGS. 5A-5D may be configured to operate with the POD connector of FIGS. 8A-8D.

As shown in FIGS. 5A-5D, a direct-connection POD receptacle 432 may include multiple components the same or similar to those of covered-connection POD receptacle 431 of FIGS. 4A-4D. In some embodiments, machine side 471 of POD receptacle 432 may include a POD connector opening 573 for receiving a POD connector that may be connected to the sensor port of a POD installed within POD receptacle 432. In some embodiments, POD connector opening 573 may include connector engagement structures 574, including, without limitation, teeth, flanges, grooves, projections, and/or the like for engaging corresponding structures of a POD connector. In some embodiments, a POD connector seat or shelf 561 may be included for engaging an external-facing side of a POD connector. In some embodiments, connector engagement structures 574 may be formed within POD connector shelf 561.

FIGS. 6A-6D illustrate an exemplary covered-connection connector in accordance with the present disclosure. More specifically, FIGS. 6A-6D depict various views of a covered-connection connector, including a side view (FIG. 6A), a top perspective view (FIG. 6B), a top view (FIG. 6C), and a bottom view (FIG. 6D).

As shown in FIGS. 6A-6D, a covered-connection connector 650 may include a body 662 having a bottom or machine end 651 and a top or POD end 652. Machine end 651 may be configured to be coupled to a machine (e.g., pressure port) and POD end 652 may be configured to be coupled to POD 332.

In some embodiments, machine end 651 may include a connector opening or fitting 654 configured to engage a connection element of a machine where connector 650 is being installed. In some embodiments, connector fitting 654 may be coupled to a corresponding machine connection element via friction fit, interference fit, press fit, twist-and-lock, threaded fit, and/or the like. In various embodiments, connector fitting 654 may be or may include a female luer fitting configured to engage a corresponding male luer fitting of a pressure port.

When connector 650 is installed on machine (e.g., coupled to a corresponding machine connection element), POD end 652 may extend away from the surface of the machine. In some embodiments, connector 650 may be coupled to machine (e.g., to a machine connection element) via a fastener, such as a set screw. For instance, machine end 651 may envelope or otherwise be arranged over a portion of a machine connection element. Machine end 651 may include a set screw threaded opening (not shown) configured to receive a set screw to engage and affix machine end 651 to the machine connection element. In this manner, connector 650 may be securely fastened to machine.

In various embodiments, POD end 652 may include a port opening 655 configured to receive sensor port 345 of POD 332. Port opening 655 and sealing element 656 may be configured to form a seal, such as via a luer fitting (e.g., luer lock, slip luer, and/or the like), between connector 650 and POD 332. For example, sensor port 345 may have an end configured as a female luer fitting and POD end 652 (via port opening 655 and sealing element 656) may form a male luer fitting. POD 332, and particularly sensor port 345, may be fluidically coupled to connector 650, for instance, at POD end 652.

In some embodiments, POD end 652 may include one or more flanges, wings, projections and/or the like 653. In various embodiments, flanges 653 may be configured to engage corresponding slots or grooves of a cover element configured to be arranged over connector 650 (see, for example, FIGS. 7A-7C and 9A-9D).

FIGS. 7A-7C illustrate an exemplary covered-connection cover in accordance with the present disclosure. More specifically, FIGS. 7A-7C depict various views of a covered-connection cover, including a side perspective view (FIG. 7A), a top perspective view (FIG. 7B), and a bottom perspective view (FIG. 7D). In general, a covered-connection cover is configured to be installed over covered-connection cover 650.

As shown in FIGS. 7A-7C, a covered-connection cover 760 may include a body 767 having a machine end 761 configured to be installed toward the machine and a POD end 762 configured to be installed toward POD 332. A connector opening 763 may be configured to be positioned over POD end 652 of connector 650. Grooves 765 may be arranged in machine end 762 that correspond with flanges 653 of connector 650. A connector shelf or seat 766 may be arranged within cover 760 for engaging a portion of connector 650. When installed, sensor port 345 of POD 332 may extend through a port opening 763 of cover.

In various embodiments, cover 760 may be coupled to machine (e.g., a pressure port of machine) and/or connector 650. For example, cover 760 may be coupled to machine (e.g., a pressure port of machine) and/or connector 650 via a fastener, such as a set screw. In some embodiments, a threaded set screw opening 764 may be arranged at or near machine end 762. Opening 764 may be threaded and configured to engage the threads of a set screw configured to couple cover 760 to machine (e.g., a pressure port of machine) and/or connector 650. For example, a set screw installed in opening 764 may engage machine and/or connector 650 to hold cover to machine and/or connector 650.

FIGS. 8A-8C illustrate an exemplary direct-connection connector in accordance with the present disclosure. More specifically, FIGS. 8A-8C depict various views of a direct-connection connector, including a side view (FIG. 8A), a top perspective view (FIG. 8B), and a bottom perspective view (FIG. 8C).

As shown in FIGS. 8A-8C, a direct-connection connector 850 may include a body 862 having a bottom or machine end 851 and a top or POD end 852. Machine end 851 may be configured to be coupled to a machine (e.g., pressure port) and POD end 852 may be configured to be coupled to POD 332.

In some embodiments, machine end 851 may include a connector opening or fitting 854 configured to engage a connection element of a machine where connector 850 is being installed. In some embodiments, connector fitting 854 may be coupled to a corresponding machine connection element via friction fit, interference fit, press fit, twist-and-lock, threaded fit, and/or the like. In various embodiments, connector fitting 854 may be or may include a female luer fitting configured to engage a corresponding male luer fitting of a pressure port.

When connector 850 is installed on machine (e.g., coupled to a corresponding machine connection element), POD end 852 may extend away from the surface of the machine. In some embodiments, connector 850 may be coupled to machine (e.g., to a machine connection element) via a fastener, such as a set screw. For instance, machine end 851 may envelope or otherwise be arranged over a portion of a machine connection element. Machine end 851 may include a set screw threaded opening (not shown) configured to receive a set screw to engage and affix machine end 851 to the machine connection element. In this manner, connector 850 may be securely fastened to machine.

In various embodiments, POD end 852 may include a port opening 855 configured to receive sensor port 345 of POD 332. Port opening 855 and sealing element 856 may be configured to form a seal, such as via a luer fitting (e.g., luer lock, slip luer, and/or the like), between connector 850 and POD 332. For example, sensor port 345 may have an end configured as a female luer fitting and POD end 852 (via port opening 855 and sealing element 856) may form a male luer fitting. POD 332, and particularly sensor port 345, may be fluidically coupled to connector 850, for instance, at POD end 852.

In some embodiments, POD end 852 may include one or more ridges, flanges, wings, projections and/or the like 877. In various embodiments, ridges 877 may be configured to engage corresponding slots or grooves of POD receptacle (e.g., connector engagement structures 574).

FIGS. 9A-9D illustrate an exemplary covered-connection POD assembly in accordance with the present disclosure. More specifically, FIGS. 9A-9D depict various views of a covered-connection POD assembly, including an exploded side view (FIG. 9A), a top perspective view (FIG. 9B), a cross-sectional side view (FIG. 9C), and a side view with a transparent POD receptacle and cover (FIG. 9D).

As shown in FIGS. 9A-9D, a covered-connection POD assembly 205 may include a connector 650, a cover 760, a POD receptacle 431, and a POD 332. In some embodiments, connector 650 is coupled to port 217 of machine 215. For example, connector 650 may establish a luer lock or other water- and/or gas-tight seal with port 217. Cover 760 may be arranged over connector 650 (see, for example, FIG. 11A). Flanges 653 may be arranged within corresponding cavities or grooves 765 of cover 760. The arrangement of flanges 653 within grooves 765 may prevent, reduce, or even eliminate unwanted movement of POD receptacle 431, such as unwanted rotation. In some embodiments, cover 760 may be coupled to connector 650 via an interference fit, twist-and-lock, one or more fasteners, such as a set screw view threaded opening 764, and/or the like.

POD receptacle 431 may be arranged over cover 760. In some embodiments, POD receptacle 431 may be coupled to connector 650 via an interference fit, twist-and-lock, one or more fasteners, such as a set screw view threaded opening 764, and/or the like. POD 332 may be installed within POD receptacle 431, for instance, arranged within POD opening 456 on POD seat 457. When installed within POD receptacle, sensor port 345 of POD 332 may form a luer lock or other water- and/or gas-tight seal with POD end 652 of connector 650.

The combination of connector 650, cover 760, and POD receptacle 431 may be rigidly affixed to machine 215. POD 332 may be easily removed and re-affixed with minimal effort. For example, pulling on POD 332 (and/or extracorporeal circuit tubing attached to POD 332 ports) may allow for removal of POD 332 from POD receptacle (e.g., breaking seal between sensor port 345 and POD end 652 of connector 650). POD 332 may be attached by simply pushing POD back 332 within POD receptacle 431 and re-establishing the coupling of sensor port 345 with POD end 652.

FIGS. 10A-10D illustrate an exemplary direct-connection POD assembly in accordance with the present disclosure. More specifically, FIGS. 10A-10D depict various views of a direct-connection POD assembly, including an exploded side view (FIG. 10A), a top perspective view (FIG. 10B), a cross-sectional side view (FIG. 10C), and a side view with a transparent POD receptacle and cover (FIG. 10D).

As shown in FIGS. 10A-10D, a direct-connection POD assembly 205 may include a connector 850, a POD receptacle 432, and a POD 332. In some embodiments, connector 850 is coupled to port 217 of machine 215. For example, connector 850 may establish a luer lock or other water- and/or gas-tight seal with port 217. POD receptacle 432 may be arranged over connector 850. In some embodiments, POD receptacle 432 may be coupled to connector 850 via an interference fit, twist-and-lock, one or more fasteners, such as a set screw view threaded opening 764, and/or the like. In some embodiments, ridges 877 of connector 850 may be arranged within corresponding grooves 574 of POD receptacle 432.

POD 332 may be installed within POD receptacle 432, for instance, arranged within POD opening 456 on POD seat 457. When installed within POD receptacle 432, sensor port 345 of POD 332 may form a luer lock or other water- and/or gas-tight seal with POD end 852 of connector 850.

The combination of connector 850 and POD receptacle 432 may be rigidly affixed to machine 215. POD 332 may be easily removed and re-affixed with minimal effort. For example, pulling on POD 332 (and/or extracorporeal circuit tubing attached to POD 332 ports) may allow for removal of POD 332 from POD receptacle (e.g., breaking seal between sensor port 345 and POD end 852 of connector 850). POD 332 may be attached by simply pushing POD back 332 within POD receptacle 432 and re-establishing the coupling of sensor port 345 with POD end 852. Accordingly, POD assemblies according to some embodiments may facilitate plug-in POD devices that are easy to install, change, remove, and/or the like for dialysis technicians, patients, caregivers, and/or the like.

FIGS. 11A-11C illustrate an exemplary POD assembly coupled to a dialysis machine in accordance with the present disclosure. More specifically, FIGS. 11A-11C depict a connection element attached to a dialysis machine (FIG. 11A), POD receptacles attached to a dialysis machine (FIG. 11B), and PODs installed within the POD receptacles (FIG. 11C).

Referring to FIG. 11A, connection elements, such as covers 760 may be arranged on an external surface of a dialysis machine 1101. Cover 760A is for an arterial POD attached to an arterial pressure port and cover 760B is for a venous POD attached to a venous pressure port. Although FIGS. 11A-11C depict a covered-connection POD assembly, embodiments are not so limited as this is for illustrative purposes within this detailed disclosure. For example, in a direct-connection configuration, one or more of covers 760A, 760B may be replaced with a direct-connection connector 850 for use with a direct-connection receptacle 432. Referring to FIG. 11B, an arterial POD receptacle 431A is connected to cover 760A and a venous POD receptacle 432B is connected to cover 760B.

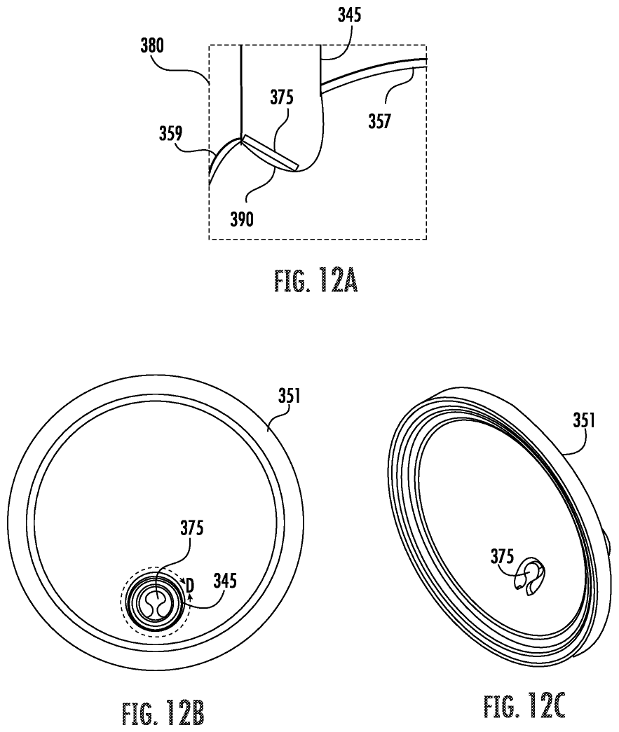

Referring to FIG. 3D, in some embodiments, POD 332 may include a flange, projection, or other element 375 configured to be pushed in (toward chamber 355) to create a dimple in membrane 357. FIGS. 12A-12C illustrate an exemplary flanged POD membrane in accordance with the present disclosure.

FIG. 12A depicts detail area 380 of FIG. 3D with flange 375 pushed in or activated to create a dimple, indent, recess, or other structure 390 in membrane 357. Flange 375 may operate as a hinged tab that may be pressed into membrane 357. Dimple 390 may operate to prevent membrane 357 from locking with the inner surface of cap 351, for instance, due to vapor lock (under positive pressure). Once dimple 390 is created, the shape may be retained in membrane 357 to prevent vapor lock between membrane 357 and the inner surface (e.g., chamber-side surface) of cap 351.

FIGS. 12B and 12C depict a top-down view and a bottom perspective view, respectively, of cap 351 showing flange 375. In some embodiments, flange 375 may be arranged at an opening of sensor port 345 with chamber 355.

In an alternative embodiment, a projection (e.g., a permanent projection that is not folded in) may be formed on the inner surface of membrane 357 that forms dimple 390. For example, when membrane 357 is installed against cap 361, the projection will push against membrane 357 and create a dimple.

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A pressure output device (POD) assembly, comprising:
a connector configured to be coupled to a measurement port arranged on an outer surface of a fluid processing system;
a POD receptacle configured to be installed on the outer surface over the connector, the POD receptacle configured to be coupled to the connector; and
a POD comprising an inlet port, an outlet port, and a sensor port, the POD configured to be removably installed within a POD seat formed in the POD receptacle, wherein the sensor port is arranged within a POD opening of the POD receptacle and coupled to the connector to fluidically couple the POD to the measurement port.

2. The POD assembly of claim 1, wherein the connector comprises:
a covered-connection connector configured to be coupled to the measurement port; and
a cover configured to be installed over the connection element.

3. The POD assembly of claim 1, wherein the covered-connection connector comprises at least one flange configured to engage at least one groove of the cover when the cover is installed over the covered-connection connector.

4. The POD assembly of claim 1, wherein the connector comprises a direct-connection connector having a POD end configured to engage the POD receptacle.

5. The POD assembly of claim 4, wherein the direct-connection connector comprises ridges configured to engage at least one engagement structure of the POD receptacle when the POD receptacle is installed over the direct-connection connector.

6. The POD assembly of claim 1, wherein the connector is configured to form a luer connection with the measurement port.

7. The POD assembly of claim 1, wherein the POD receptacle is configured to be coupled to the connector via a set screw fastener.

8. The POD assembly of claim 1, wherein the POD is configured to be installed within the POD receptacle by pushing the POD within the POD opening toward the fluid processing system until the POD is seated in the POD seat.

9. The POD assembly of claim 1, wherein the POD forms an interference fit with the POD seat when installed in the POD receptacle.

10. The POD assembly of claim 1, wherein the outlet port is arranged in a top position, vertically above a bottom position of the inlet port.

11. The POD assembly of claim 10, wherein fluid flow within a chamber is in a rotary fluid flow rotating from the inlet port to the outlet port.

12. The POD assembly of claim 1, wherein the fluid processing device is a hemodialysis (HD) machine.

13. The POD assembly of claim 1, wherein the measurement port is a pressure port of a hemodialysis (HD) machine.

14. The POD assembly of claim 13, wherein the POD is fluidically coupled with a pressure sensor of a hemodialysis (HD) machine to provide pressure readings for arterial or venous blood flowing through an extracorporeal circuit.

15. The POD assembly of claim 1, wherein the POD comprises a flange configured to engage a membrane of the POD to form a dimple in the membrane to prevent vapor lock between the membrane and an inner surface of the POD.

16. A method of measuring pressure of blood flowing through an extracorporeal circuit of a dialysis machine, the method comprising:
installing a pressure output device (POD) comprising an inlet port, an outlet port, and a sensor port in a POD assembly, the POD assembly comprising:
a connector configured to be coupled to a pressure port arranged on an outer surface of the dialysis machine; and
a POD receptacle configured to be installed on the outer surface over the connector, the POD receptacle configured to be coupled to the connector;
wherein the POD is configured to be removably installed within a POD seat formed in the POD receptacle, wherein the sensor port is arranged within a POD opening of the POD receptacle and coupled to the connector to fluidically couple the POD to the pressure port; and
measuring fluid pressure using a pressure sensor detecting movement of a membrane of the POD resulting from a flow of the blood through the POD.

17. The method of claim 16, wherein the connector comprises:
a covered-connection connector configured to be coupled to the measurement port; and
a cover configured to be installed over the connection element.

18. The method of claim 16, wherein the connector comprises a direct-connection connector having a POD end configured to engage the POD receptacle.

19. The POD assembly of claim 1, wherein the outlet port is arranged in a top position, vertically above a bottom position of the inlet port.

20. The POD assembly of claim 10, wherein fluid flow within a chamber is in a rotary fluid flow rotating from the inlet port to the outlet port.
